(19) United States
(12) Patent Application Publication
(10) Pub. No.: US 2024/0173223 A1
(43) Pub. Date: May 30, 2024

(54) NEW TOPICAL COMPOSITION FREE OF SULFATE DERIVED SURFACTANTS

(71) Applicant: PIERRE FABRE DERMO-COSMETIQUE, BOULOGNE-BILLANCOURT (FR)

(72) Inventors: Fernanda CHAVES, GAVEA RIO DE JANEIRO (BR); Juliana AMADO, CATETE RIO DE JANEIRO (BR); Anne Laure GAUDRY, BALMA (FR); Danielle BORHER, TIJUCA RIO DE JANEIRO (BR)

(73) Assignee: PIERRE FABRE DERMO-COSMETIQUE, BOULOGNE-BILLANCOURT (FR)

(21) Appl. No.: 18/282,131

(22) PCT Filed: Mar. 15, 2022
(86) PCT No.: PCT/EP2022/056689

§ 371 Date: Sep. 14, 2023

(30) Foreign Application Priority Data
Mar. 15, 2021 (EP) 21162677.5

(51) Int. Cl. A61K 8/23; A61K 8/42; A61K 8/44; A61K 8/60; A61K 8/9789; A61Q 5/00; A61Q 19/10

(52) U.S. Cl. CPC A61K 8/23; A61K 8/42; A61K 8/44; A61K 8/604; A61K 8/9789; A61Q 5/006; A61Q 19/10

(57) ABSTRACT

The present disclosure relates to topical compositions comprising N-ethyl-lauroyl-arginate, an antimicrobial agent, that may be particularly useful as preservative and active ingredient in personal care products, such as cleansing body or face compositions or shampoos and conditioners.

### FIELD OF THE INVENTION

The invention relates to topical compositions comprising N-ethyl-lauroyl-arginate, an antimicrobial agent, that may be particularly useful as preservative and active ingredient in personal care products, such as cleansing body or face compositions or shampoos and conditioners.

### BACKGROUND OF THE INVENTION

Dandruff is a common and chronic dermatological condition that affect the seborrheic areas of the body. It is restricted to the scalp, and involves itchy, flaking skin without visible inflammation. Flaking in dandruff is usually white-to-yellowish, and may be oily or dry. Various intrinsic and environmental factors, such as sebaceous secretions, skin surface fungal colonization, individual susceptibility, and interactions between these factors, all contribute to the pathogenesis of dandruff. Genetic, biochemical studies and investigations in animal models further provided insight on the pathophysiology and strategies for better treatment.

It is estimated that dandruff affects approximately 50% of the general adult population worldwide. It is also more prevalent in males than females. Dandruff starts at puberty, reaches peak incidence and severity at the age of about 20 years, and becomes less prevalent among people over 50. Incidence varies between different ethnic groups: in a study in the U.S. and China, dandruff prevalence was 81-95% in African Americans, 66-82% in Caucasians, and 30-42% in Chinese. Besides physical discomfort such as itching, dandruff is socially embarrassing and negatively impacts patients' self-esteem.

Multiple predisposing factors have been identified in the pathogenesis of dandruff. The presence and abundance of Malassezia yeast, host epidermal conditions and sebaceous secretion, combined with various other factors, and interactions between these factors, determine an individual's susceptibility to dandruff. In a likely scenario, there may be aberrant epidermal barrier function due to genetic predisposition, and excessive or altered sebum composition that provides a favorable milieu for Malassezia colonization. N-ethyl-lauroyl-arginate, also known as LAE, is a white cationic solid, non-toxic preservative-like with a high antimicrobial activity. It is synthetized from:
L-arginine, a non-essential aminoacid present in many food sources;
Lauric acid, a medium chain fatty acid present mainly in coconut oil and palm seed oil; and
ethanol, an organic substance obtained by fermentation of sugars. LAE has the following structure:

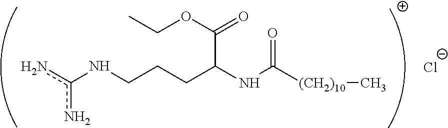

LAE is an antimicrobial agent that acts on the cell membranes and the cell cytoplasm. Thanks to its antimicrobial property, LAE may be used as preservative and represents an alternative of choice to replace preservatives that may be of safety concerns for human use or for the environment, such as for example triclosan and isothiazolinones. Thanks to its antimicrobial properties, LAE may also be used as an active ingredient in soaps, anti-dandruff shampoos and deodorants. LAE is a readily biodegradable material and is a non-irritating and non-sensitizing compound. When used in antidandruff products, it may represent an ecofriendly alternative to salicylic acid, zinc pyrithione and piroctone olamine while being as effective than these compounds against Malassezia furfur.

Thus, the use of LAE appears very attractive in the personal care products industry. However, formulating compounds comprising LAE appears very challenging. LAE is a cationic molecule which is incompatible with most of anionic surfactants commonly used in the industry, such as alkyl phosphates and carboxylates.

Therefore, a need remains for readily formulating topical compositions that comprise LAE and which exhibit good storage stability, suitable viscosity for the intended use, optimal environmental safety profile while at the same time being gentle to the skin/hair, delivering good working properties (e.g., efficient washing or cleansing) and minimizing the risk of undesirable interactions between components.

### SUMMARY OF THE INVENTION

The invention relates to a topical composition comprising:
(a) from 0.05 to 1%, preferably from 0.09 to 0.8%, by weight of N-ethyl-lauroyl-arginate relative to the total weight of the composition;
(b) a surfactant system comprising:
   (b1) one or more amino acid-based anionic surfactants;
   (b2) one or more alkyl glucoside non-ionic surfactants;
   (b3) one or more amphoteric surfactants different from betaine amphoteric surfactants;
(c) one or more physiologically acceptable carriers and/or adjuvants; wherein the surfactant system does not comprise more than six surfactants.

Further aspects of the invention are as disclosed herein and in the claims.

### FIGURE

FIG. 1 represents the values of sinking time for the Treatment Group when compared to the Natural group-mean value (example 4).

### DEFINITIONS

The term “about” means in the context of the present invention that the concerned value may be lower or higher by 10%, especially by 5%, in particular by 1%, than the indicated value. It encompasses the indicated value and values that may be lower or higher by 10%, especially by 5%, in particular by 1%, than the indicated value. As a matter of examples, when a range is said to vary from about X to about Y, it includes the range from X to Y and optionally values that may be lower by 10%, especially by 5%, in particular by 1%, than X and values that may be higher by 10%, especially by 5%, in particular by 1% than Y.

Percentage by weight, percentage by mole and percentage by volume are respectively abbreviated herein as wt. %, mol. % and vol. %.

### DESCRIPTION OF THE INVENTION

It has now been discovered that compositions comprising a surfactant system as disclosed herein allow providing topical compositions which are storage stable, visually appealing (not cloudy), which exhibit a viscosity that is adapted to the intended uses while at the same time being gentle to the skin/hair, having good working properties (e.g., good washing/cleansing and conditioning properties) and delivering a nice consumer experience. When the compositions are used as washing/cleansing compositions (e.g., shampoos, shower gels), they allow delivering a rich, creamy and abundant foam resulting in a pleasant sensation. Advantageously, these effects may be obtained while using a reasonable number of components in the surfactant system which minimizes the risk of undesirable interactions. The surfactant system does not comprise more than six surfactants, or no more than five surfactants, or no more than four surfactants or no more than three surfactants. For sake of clarity, it is specified that in no instance LAE is considered as a component of the surfactant system.

The topical composition of the present invention comprises:
(a) from 0.05 to 1%, preferably from 0.09 to 0.8%, more preferably from 0.1 to 0.5%, by weight of N-ethyl-lauroyl-arginate relative to the total weight of the composition;
(b) a surfactant system comprising:
   (b1) one or more, preferably only one, amino acid-based anionic surfactants;
   (b2) one or more, preferably only one, alkyl glucoside non-ionic surfactants;
   (b3) one or more, preferably only one, amphoteric surfactants different from betaine amphoteric surfactants;
(c) one or more physiologically acceptable carriers and/or adjuvants;
wherein the surfactant system does not comprise more than six surfactants.

By “topical composition” as used herein, it is meant a composition for application to the skin (e.g., body, face, scalp), mucous membranes and/or hair of humans. The topical composition may be a cosmetic topical composition or a pharmaceutical/dermatological topical composition.

By “cosmetic topical compositions” as used herein, it is meant a skin and/or hair care composition.

Topical compositions, such as topical skin and/or hair care compositions, are generally used to wash, cleanse, protect, moisturize the skin and/or hair and/or to treat the skin and/or hair, i.e., to administer beneficial/active agents in order to improve the condition of the skin and/or hair to which they are applied. Thus, the topical composition may further comprise active agents.

The topical compositions of the invention may be in any suitable form for skin and/or hair care. For instance, the topical composition may be in the form of a solution, micellar solution, lotion, emulsion, suspensions, cream, ointment, serum, mask, foam or gel. Solution, lotion, foam and gel may be preferred. The choice of suitable carriers and adjuvants as described herein will largely depend on the selected form of the composition.

In some embodiments, the topical composition is a face or body cleansing composition.

The topical cleansing composition is generally a rinse-off composition. By “rinse-off composition”, it is meant a composition that is applied to the skin or hair for a short period of time (few seconds or minutes) and then rinsed off with water.

In some embodiments, the topical composition is a hair washing composition (i.e., a shampoo) or a conditioner. In particular, thanks to the presence of LAE, the topical composition may be particularly useful as an antidandruff composition. When the topical composition is an antidandruff composition, it may comprise one or more further antidandruff agents as disclosed herein.

When the topical composition is a shampoo, it was found to efficiently protect the hair fiber and reduce the occurrence of hair damages. It efficiently cleanses the scalp while being gentle to the scalp (reduces itchiness, erythema), it efficiently reduces scalp oiliness and efficiently contributes to the reduction of dandruff flakes with effects that are immediately apparent for the consumers (after the first use of the shampoo).

In some embodiments, the antidandruff topical composition allows a beneficial action on the scalp while protecting hair fibers. In particular, it efficiently cleanses the scalp and allows a reduction of erythema, dandruff flakes, scalp itchiness.

The topical composition preferably has a pH that ranges from 4.5 to 6.5, e.g., from 5.0 to 6.5.

The topical cleansing composition is preferably an aqueous-based composition, i.e., the topical cleansing composition is not an oil-based composition, nor an emulsion-type composition nor a solvent-based composition (e.g., alcohol-based composition).

In some embodiments, the topical cleansing compositions are monophase compositions.

The topical cleansing composition is preferably liquid. Preferably, the topical cleansing composition has a viscosity at ambient temperature (25° C.) that varies within a broad range, for example a viscosity ranging from 500 to 3500 cPs, preferably from 700 to 1600 cPs. The viscosity is generally measured at 25° C., using a viscosimeter Brookfield DV1RV.

In some embodiments, the topical composition comprises:
(a) from 0.05 to 1%, preferably from 0.09 to 0.8%, more preferably from 0.1 to 0.5%, by weight of N-ethyl-lauroyl-arginate relative to the total weight of the composition;
(b) from 8 to 30%, preferably from 10 to 20%, by weight of a surfactant system relative to the total weight of the composition, the surfactant system comprising:
   (b1) one or more, preferably only one, amino acid-based anionic surfactants;
   (b2) one or more, preferably only one, alkyl glucoside non-ionic surfactants;
   (b3) one or more, preferably only one, amphoteric surfactants different from betaine amphoteric surfactants;
(c) one or more physiologically acceptable carriers and/or adjuvants;
wherein the surfactant system does not comprise more than six surfactants.

It is to be understood that any surfactants present in the topical composition are part of the surfactant system.

Components of the topical compositions are as disclosed herein below.

Surfactant System

The surfactant system comprises:
(b1) one or more, preferably only one, amino acid-based anionic surfactants;
(b2) one or more, preferably only one, alkyl glucoside non-ionic surfactants;
(b3) one or more, preferably only one, amphoteric surfactants different from betaine amphoteric surfactants.

The surfactant system does not comprise more than six surfactants.

“Betaine amphoteric surfactants” as used herein designate betaines, N-alkylamidobetaines and derivatives thereof (e.g., sulfobetaine, N-alkylamidosulfobetaines).

In some embodiments, the surfactant system is free from sulfate-based anionic surfactants and/or from cationic surfactants and/or from betaine amphoteric surfactants.

“Sulfate-based surfactants” as used herein designate any anionic surfactant comprising a sulfate function. Non-limitary examples of sulfate-based surfactants include alkyl sulfates, alkyl ether sulfates, alkylamido ether sulfates, alkylaryl polyether sulfates and monoglyceride sulfates.

“Cationic surfactants” as used herein designate compounds that carry a positive charge on their hydrophilic end.

In some embodiments, the surfactant system does not comprise further surfactants.

Hence, in some embodiments, the surfactant system consists of:
(b1) one or more, preferably only one, amino acid-based anionic surfactants;
(b2) one or more, preferably only one, alkyl glucoside non-ionic surfactants;
(b3) one or more, preferably only one, amphoteric surfactants different from betaine amphoteric surfactants.

In some embodiments, the surfactant system consists of:
(b1) only one amino acid-based anionic surfactant;
(b2) only one alkyl glucoside non-ionic surfactant;
(b3) only one amphoteric surfactant different from betaine amphoteric surfactants.

It was found that a surfactant system comprising a number of surfactants as low as three surfactants allows providing a topical composition comprising LAE which is storage stable, visually appealing (not cloudy), which exhibits a viscosity that is adapted to the intended uses while at the same time being gentle to the skin/hair, having good working properties (e.g., good washing/cleansing and conditioning properties) and delivering a nice consumer experience.

In some embodiments, the surfactant system comprises, or consists of:
(b1) a total amount of amino acid-based anionic surfactants ranging from 0.09 to 12%, preferably from 2 to 10%, more preferably from 5 to 8% by weight relative to the total weight of the composition;
(b2) a total amount of alkyl glucoside non-ionic surfactants ranging from 1 to 30%, preferably from 2 to 15%, more preferably from 3 to 8% by weight relative to the total weight of the composition;
(b3) a total amount of amphoteric surfactants ranging from 1.5 to 10%, preferably from 2 to 8%, more preferably from 2 to 5% by weight relative to the total weight of the composition.

The surfactant system does not comprise more than six surfactants.

In some embodiments, the weight ratio total amount of anionic surfactant/total amount of non-ionic surfactant is equal or superior to 1, preferably equal or superior to 1.4.

In some embodiments, the weight ratio total amount of anionic surfactant/total amount of non-ionic surfactant is equal or inferior to 3, preferably equal or inferior to 2.5 and more preferably equal or inferior to 1.5.

In some embodiments, the weight ratio total amount of anionic surfactant/total amount of non-ionic surfactant ranges from 1 to 2, preferably from 1 to 1.8 and more preferably 1 to 1.5.

In some embodiments, the weight ratio total amount of anionic surfactant/total amount of amphoteric surfactant is equal or superior to 1.7, preferably equal or superior to 1.9. In some embodiments, the ratio total amount of anionic surfactant/total amount of amphoteric surfactant is equal or inferior to 4, preferably equal or inferior to 2.5.

In some embodiments, the weight ratio total amount of non-ionic surfactant/total amount of amphoteric surfactant is equal or superior to 1, preferably equal or superior to 1.2. In some embodiments, the ratio total amount of non-ionic surfactant/total amount of amphoteric surfactant is equal or inferior to 4, preferably equal or inferior to 3 or to 2.

In some embodiments, the weight ratio total amount of anionic surfactant/total amount of non-ionic surfactant is equal or superior to 1 and the weight ratio total amount of non-ionic surfactant/total amount of amphoteric surfactant is equal or superior to 1.

The surfactants used in the surfactant system may be as disclosed herein below.

The surfactants system provides cleaning benefits, lather properties and rheology properties to the topical compositions. The surfactant system is mild, which means that the surfactants provide sufficient cleaning but do not overly dry the skin or the hair.

Amino Acid-Based Anionic Surfactants

Amino acid-based anionic surfactants are a widely known class of biocompatible and biodegradable surfactants. They may result from the combination of natural fatty acids, alcohols and amines with different amino acid head groups through ester and amide linkages, e.g., acyl glutamate, acyl, sarcosinate, acyl alaninate, acyl glycinate, acyl apple amino acids based anionic surfactants.

Examples of suitable amino acid-based anionic surfactants include, but are not limited to, sodium lauroyl glutamate, sodium lauroyl glycinate, sodium cocoyl glutamate, TEA-cocyl glutamate, disodium cocoyl glutamate, sodium cocoyl glycinate, potassium cocoyl glycinate, sodium cocoyl alaninate, sodium myristoyl glutamate, disodium myristoyl glutamate, sodium sarcosinate, potassium cocoyl glycinate, sodium cocoyl alaninate, sodium cocoyl apple amino acids and mixtures thereof.

The amount of amino acid-based anionic surfactants in the composition may range from 0.09 to 12%, preferably from 2 to 10%, more preferably from 5 to 8% by weight, relative to the total weight of the composition.

In some embodiments, the amino acid-based surfactant in the composition is disodium cocoyl glutamate. Disodium cocoyl glutamate delivers excellent conditioning effect on the hair, leaving it silky and not damaging to the fiber.

Alkyl Glucoside Non-Ionic Surfactants

Alkyl glucosides are a class of non-ionic surfactants consisting of a hydrophilic saccharide moiety (e.g., glucose, fructose, mannose, galactose, and the like) and a hydrophobic fatty alkyl chain. They are synthesized from renewable raw materials and have excellent ecotoxicological profiles and are readily biodegradable. The term “alkyl glucoside” is intended to encompass alkyl monoglucosides, alkyl oligoglucosides and alkyl polyglucosides. The term “alkyl” in alkyl glucosides preferably refers to hydrocarbon chains comprising from 8 to 20 carbon atoms.

Examples of suitable alkyl glucoside surfactants include, but are not limited to decyl glucoside, arachidyl glucoside, mixture of C₁₂-C₂₀ alkyl glucoside, mixture of C₈-C₁₆ alkyl glucoside, cetearyl glucoside, ethyl glucoside, lauryl glucoside, coco glucoside and caprylyl/capryl glucoside.

The amount of alkyl glucoside non-ionic surfactants in the composition may range from 1 to 30%, preferably from 2 to 15%, more preferably from 3 to 8% by weight, relative to the total weight of the composition.

In some embodiments, the alkyl glucoside non-ionic surfactants in the composition are selected from the group consisting of decyl glucoside, C₈-C₁₆ alkyl glucoside, lauryl glucoside, and coco glucoside. Coco glucoside, for example Plantacare®818UP by BASF, provide excellent performance in foaming, cleaning and mildness, which makes it ideal for mild shampoo formulations.

Amphoteric Surfactants

The amphoteric surfactants different from betaine amphoteric surfactants may include glycine derivatives, sultaines, alkylpolyaminocarboxylates, alkylamphoacetates and alkylamphodiacetates.

In some embodiments, the amphoteric surfactant is selected from the group consisting of sultaines (e.g., cocamidopropyl hydroxysultaine), alkylpolyaminocarboxylates (e.g., carboxymethyl tallow polypropylamine, sodium cocoyipolyamino carboxylate, sodium carboxymethylolpropylamine and sodium carboxymethyl oleyl polypropylamine), alkylamphoacetates and derivatives (e.g., disodium cocoamphoacetate, sodium cocoamphoacetate and disodium lauroamphoacetate, lauroamphoacetate), alkylamphodiacetates and derivatives (e.g., disodium cocoamphodiacetate, lauroamphodiacetate), glycine derivatives (e.g., cocoamphopolycarboxyglycinate and N-cocoylglycinate) and mixtures thereof.

The amount of amphoteric surfactants different from betaine amphoteric surfactants in the composition may range from 1.5 to 10%, preferably from 2 to 8%, more preferably from 2 to 5% by weight, relative to the total weight of the composition.

In some embodiments, the amphoteric surfactants in the composition are selected from the group consisting of cocoamphoacetate, cocoamphodiacetate, lauroamphoacetate, lauroamphodiacetate and mixtures thereof.

In some embodiments, the amphoteric surfactant in the composition is sodium cocoamphoacetate. Sodium cocoamphoacetate exhibits good skin/hair compatibility.

Physiologically Acceptable Carriers and/or Adjuvants

The compositions of the invention being intended for topical application, the one or more carriers and/or excipients should be physiologically acceptable.

The term “physiologically acceptable” means compatible with the skin (e.g., body, face, eyelids), mucous membranes (e.g., lips) and keratinous materials, i.e., it does not induce undue toxicity, incompatibility, instability, irritation, allergic response, or the like.

The choice of suitable carriers and adjuvants will largely depend on the selected form of the topical composition.

Carriers

The topical compositions may comprise from about 10% to about 90%, e.g., from about 10% to about 70% or from about 15% to about 60% by weight of the composition of one or more carriers.

Though the physiology acceptable carrier could be an organic solvent (e.g., propylene glycol, polyethylene glycol, polypropylene glycol, glycerol, 1,2,4-butanetriol, sorbitol esters, 1,2,6-hexanetriol, ethanol, and mixtures thereof), a silicone solvent, oils, lipids and/or waxes, the physiologically acceptable carrier of the topical compositions is typically water. Preferably, water is used as the sole carrier. The topical composition is then preferably an aqueous-based composition, i.e., the topical composition is not an oil-based composition nor an emulsion-type composition nor a solvent-based composition (e.g., alcohol-based composition). Hence, in preferred embodiments, the topical composition is free from oily phase and from oil-containing components.

Adjuvants

The topical compositions may also comprise adjuvants that are common in the cosmetic or dermatological field, such as thickeners, gelling agents, preservatives, emulsifiers (e.g., mono- and di-glycerides, fatty alcohols, polyglycerol esters, propylene glycol esters, sorbitan esters). The amounts of these various adjuvants are those conventionally used in the field under consideration, for example from about 0.0001% to about 30%, or from about 0.0001% to about 20%, by weight relative to the total weight of the topical composition, or from about 0.01% to about 20% relative to the total weight of the topical composition.

The topical composition may comprise thickeners, such as cellulose derivatives (e.g., hydroxymethyl cellulose and hydroxypropyl cellulose), starches and starch derivatives, acrylic acid and acrylate polymers and copolymers (e.g. carbomer), polyalkyl glycol (e.g., Ceteareth 60 myristyl glycol), polyethylene glycol derivatives (e.g., PEG-120 Methyl Glucose Dioleate, PEG-150 (Di)stearate), polyoxyethylene derivatives (e.g., Polysorbate 80) Ammonium Acryloyldimethyltaurate/VP Copolymer, natural gums (e.g., xanthan gum, scleroglucan gum and/or carrageenan gum) and salts (e.g., sodium chloride). In some embodiments, the thickener is Ceteareth 60 myristyl glycol.

The topical composition may comprise further preservatives, such as benzoic acid or salt thereof, benzyl alcohol, sorbic acid or salt thereof, ureas (e.g., imidazolidinyl urea, diazolidinyl urea), parabens, dehydroacetic acid, sodium dehydroacetate, PHMB (polyhexamethylene biguanide), phenoxyethanol, ethylhexylglycerol, salicylic acid or salt thereof and sodium benzoate.

Active Agents

The topical composition may further comprise active agents. Active agents are agents that further improve the condition of the skin/hair to which they are applied. Any active agents known for use in hair care or personal care products may be used provided they are physically and chemically compatible with the components of the composition as described herein. The amounts of these active agents are those conventionally used in the field under consideration, for example from about 0.0001% to about 20%, or from about 0.0001% to about 15%, by weight relative to the total weight of the topical composition, or from about 0.01% to about 15% relative to the total weight of the topical composition.

Suitable active agents may be keratolytic agents. Examples of keratolytic agents include but are not limited to succinic acid, alpha hydroxy acids (e.g., salicylic acid, lactic acid) and mixtures thereof.

Suitable active agents may be anti-seborrheic agents and/or pore refiners. Examples of anti-seborrheic agents include but are not limited to 2,3-dihydroxypropyl dodecanoate, sabal extract, pumpkin seed oil, extract of urtica dioic and combinations thereof, preferably 2,3-dihydroxypropyl dodecanoate. Examples of pore refiners include but are not limited to Lens esculenta seed extract.

Suitable active agents may be antidandruff agents. Examples of antidandruff agents include but are not limited to sodium shale oil sulfonate, tea tree oil, piroctone olamine, ciclopiroxolamine, zinc pyrithione and mixtures thereof. Sodium shale oil sulfonate is a sulfonate salt of a volatile, highly sulphuric shale oil fraction in watery solution. It has anti-microbial properties and it is mainly used for anti-dandruff shampoo. Tea tree oil, also known as melaleuca oil, is an essential oil that is obtained from the leaves of the Australian tea tree. Tea tree oil is well-known to be used due its antibacterial activity. It is commonly used to treat acne, nail fungus and dandruff, for example. Preferably, the antidandruff agent is a combination of sodium shale oil sulfonate and tea tree oil.

Suitable active agents may be hair fiber benefit agents, such as sodium dilauramidoglutamide lysine (Pellicer LB 30G by Asahi Kasei Chemicals). Sodium dilauramidoglutamide lysine is a multifunctional plant derived ingredient used in shampoos.

Suitable active agents may be cooling agents. Examples of cooling agents include but are not limited to carboxamides, cyclohexyl derivatives, cyclohexanol derivatives, menthol derivatives (e.g., menthol or menthyl lactate or menthone glycerin acetal), eucalyptol, tea tree oil, eucalyptus oil, and mixtures thereof.

Further non-limiting examples of suitable active agents include perfumes or fragrances, moisturizers, vitamins or nutrients, plant extracts.

In some embodiments, the topical composition comprises, or consists of, the following components (% by weight are expressed relative to the total weight of the composition):

| | wt % |
|---|---|
| LAE | 0.09-0.8 |
| one or more, preferably only one, amino acid- | 0.09-12.0 |
| based anionic surfactants | |
| one or more, preferably only one, alkyl glucoside | 1.0-30.0 |
| non-ionic surfactants | |
| one or more, preferably only one, amphoteric | 1.5-10.0 |
| surfactants different from betaine amphoteric | |
| surfactants | |
| Thickening agent | 0.5-4.0 |
| Optional active agents | 1.0-10.0 |
| Water | qsp 100 |

In some embodiments, the topical composition comprises, or consists of, the following components (% by weight are expressed relative to the total weight of the composition):

| | wt % |
|---|---|
| LAE | 0.09-0.8 |
| Disodium cocoyl glutamate (anionic surfactant) | 0.09-12.0 |
| Decyl glucoside (non ionic surfactant) | 1.0-30.0 |
| Sodium cocoamphoacetate (amphoteric surfactant) | 1.5-10.0 |
| Ceteareth-60 myristyl glycol (non-ionic thickening | 0.5-4.0 |
| agent) | |
| Optional active agents | 1.0-10.0 |
| Water | qsp 100 |

Methods and Uses

The topical composition may be used for washing or cleansing the skin or the hair.

Hence, the invention relates to the cosmetic use of a topical composition as disclosed herein for washing or cleansing the skin or the hair.

In some embodiments, the invention relates to the use of a topical composition for the treatment of dandruff while protecting hair fibers.

The cosmetic use comprises applying an effective amount of a topical composition as described herein to an individual in need thereof, in particular to the skin or hair.

The invention also relates to a cosmetic (non-therapeutic) method for washing or cleansing the skin or hair which comprises the application of an effective amount of a topical composition as described herein to an individual in need thereof.

When the topical composition is a shampoo, the cosmetic method for washing the hair comprises (a) providing a topical composition as disclosed herein; (b) applying the topical composition to the hair; (c) emulsifying with water; and (d) rinsing the topical composition from the hair.

In some embodiments, the invention relates to the use of a topical composition for washing the hair while preventing dryness.

In some embodiments, the invention relates to the use of a topical composition for washing the hair while increasing softness and/or shining.

In some embodiments, the invention relates to the use of a topical composition for washing the hair while preventing tangling and/or split ends.

The invention also relates to a method for treating dandruff which comprises the application of an effective amount of a topical composition as described herein to an individual in need thereof.

More specifically, the (cosmetic or therapeutical) method for treating dandruff comprises (a) providing a topical composition as disclosed herein; (b) applying the topical composition to the hair; (c) emulsifying with water; and (d) rinsing the topical composition from the hair. When the topical composition is a body or face cleansing composition, the method for cleansing the body of face comprises (a) providing a topical composition as disclosed herein; (b) applying the topical composition to the body or face; (c) optionally emulsifying with water; and (c) rinsing the topical composition from the body or face.

Embodiments of the present invention will now be described by way of the following examples which are provided for illustrative purposes only, and not intended to limit the scope of the disclosure.

### EXAMPLES

### Example 1: Stability Study

The stability of various compositions was compared. The formulation of tested compositions are presented in tableau 1 (% by weight are expressed relative to the total weight of the composition).

INV designates a composition according to the present invention.

C1, C2, C3 and C4 designate compositions not in accordance with the present invention.

Stability assessment was performed by visual observations at 25° C.

TABLE 1
formulations of the tested compositions.

| | INV | C1 | C2 | C3 | C4 |
|---|---|---|---|---|---|
| | wt % | wt % | wt % | wt % | wt % |
| LAE | 0.1 | 0.1 | 0.1 | 0.1 | 0.1 |
| Disodium cocoyl glutamate | 6.0 | 6.0 | — | 6.0 | 6.0 |
| (anionic surfactant) | | | | | |
| disodium coco-glucoside | — | 2.4 | — | — | — |
| citrate (anionic surfactant) | | | | | |
| Sodium lauroyl sarcosinate | — | — | 4.5 | — | — |
| (anionic surfactant) | | | | | |
| Coco-glucoside (non ionic | 4.2 | — | — | — | 2.7 |
| surfactant) | | | | | |
| Decyl glucoside (non ionic | — | 5.3 | — | — | — |
| surfactant) | | | | | |
| Cocamide | — | — | 1.5 | — | — |
| monoisopropanolamide | | | | | |
| (non ionic) | | | | | |
| Sodium cocoamphoacetate | 3.1 | — | 4.8 | 1.5 | — |
| (amphoteric surfactant) | | | | | |
| Lauryl betaine (amphoteric | — | — | — | 4.5 | 3.8 |
| surfactant) | | | | | |
| Antidandruff agent | 0.9 | 0.9 | 0.9 | 0.9 | 0.9 |
| Keratolytic agent | 2.0 | 2.0 | 2.0 | 2.0 | 2.0 |
| Hair benefit agent | 0.03 | 0.01 | 0.03 | 0.03 | 0.03 |
| Ceteareth-60 (non-ionic | 3.0 | — | 3.0 | 3.0 | 3.0 |
| thickening agent) | | | | | |
| glyceryl caprylate/caprate | — | — | — | — | — |
| (non-ionic thickening agent) | | | | | |
| Water | qsp 100 | qsp 100 | qsp 100 | qsp 100 | qsp 100 |

The results are presented in table 2.

TABLE 2
results of stability tests

| | Appearance | Stability |
|---|---|---|
| IIV | Translucid, | Conform in all temperatures (5° C., |
| | dark yellow | 25° C. and 40° C.) for 3 months |
| C1 | Opaque | Phase separation in 24 hours at |
| | yellow | room temperature (25° C.) |
| C2 | Opaque | Phase separation in 1 week at |
| | yellow | room temperature (25° C.) |
| C3 | Opaque | Phase separation after 1 week |
| | yellow | at room temperature (25° C.) |
| | | Addition of thickening agent did |
| | | not improve the stability |
| C4 | Opaque | Phase separation in 24 hours at |
| | yellow | room temperature (25° C.) |

INV was found to be translucid and stable for at least three months at the tested temperatures and exhibited a viscosity in an acceptable range (700-1600 cPs—Viscosity was measured with a Brookfield viscosimeter DV1 RV at 25° C.) whereas C1 to C4 were found to be instable.

### Example 2: Antidandruff Shampoo

An antidandruff shampoo having the formulation presented in table 3 was prepared (% by weight are expressed relative to the total weight of the composition).

TABLE 3
Antidandruff formulation.

| Ingredients | wt % |
|---|---|
| LAE | 0.1 |
| Disodium cocoyl glutamate | 6.0 |
| (anionic surfactant) | |
| Coco-glucoside (non ionic | 4.2 |
| surfactant) | |
| Sodium cocoamphoacetate | 3.1 |
| (amphoteric surfactant) | |
| Ictasol (antidandruff | 0.8 |
| agent) | |
| Tea tree oil (antidandruff | 0.1 |
| agent) | |
| Salicylic acid (keratolytic | 2.0 |
| agent) | |
| Sodium dilauramidoglutamide | 0.03 |
| lysine (hair benefit agent) | |
| Ceteareth-60 (non-ionic | 3.0 |
| thickening agent) | |
| Polyquaternium 7 | 0.31 |
| (conditioning agent) | |
| Menthyl lactate (cooling | 1.0 |
| agent) | |
| Citric acid | Qsp pH |
| | 5.5-6.5 |
| Water | 75.6 |

### Example 3: Topical Cleansing Composition for Hair or Skin

A topical cleansing composition having the formulation presented in table 4 was prepared (% by weight are expressed relative to the total weight of the composition).

TABLE 4
Topical cleansing formulation.

| | |
|---|---|
| LAE | 0.1-0.8 |
| Disodium cocoyl glutamate | 0.09-12.0 |
| (anionic surfactant) | |
| Decyl glucoside (non ionic | 1.0-30.0 |
| surfactant) | |
| diSodium cocoamphodiacetate | 1.5-10.0 |
| (amphoteric surfactant) | |
| Ceteareth-60 myristyl glycol | 0.5-4.0 |
| (non ionic thickening agent) | |
| Citric acid | Qsp pH |
| | 5.0-6.0 |
| Water | Qsp 100 |

### Example 4: Hair Hydrophobicity Study (Float Test—Hair Fiber Protection)

The purpose of this study was to assess the effect of the hair hydrophobicity after being treated with a cosmetic product by means of a float Test. This test measures how porous hair is. Healthy hair is fairly solid, while damaged hair shafts absorb liquid quickly because they are weak.

The product used in the following tests is a shampoo according to example 2.

Twenty tresses were prepared from double bleached Caucasian hair weighing 5.0 g each and 25 cm long. All tresses underwent a standard pre-cleaning process with 10% Sodium lauryl ether sulfate (SLES) solution for 1 minute then rinsed with running water. Tresses were then dried before tests.

The hair tresses were submitted to the following treatments:

| Treatments | Study Code |
|---|---|
| Untreated tresses | Natural |
| 1 application of SLES 10% | CTRL_1ap |
| 1 application of shampoo | T01_1ap |
| 5 applications of shampoo | T01_5ap |

Treatments:
Control Group (CTRL)
a) Wet the tress for 20s and remove excess water.
b) Apply 1.0 g of SLES10% and rub the tress for 60 seconds. Rinse the tress for 30 seconds and remove excess water.
Treatment Groups
a) Wet the tress for 20s and remove excess water.
b) Apply 1.0 g of Shampoo and rub the tress for 60 seconds. Rinse the tress for 30 seconds and remove excess water.
Natural Group
No treatment

After the application of the products, the tresses were dried in a controlled environment (22±2° C., 55±5% Relative Humidity) for 24 hours. After drying, the tresses were placed individually in a 40×40×60 cm pool with 90 liters of water. While they were gently released on the water surface, the tresses were filmed until they sank completely. By analyzing the videos obtained using an image analysis program, the time in seconds was determined for the tresses to sink completely.

Results:

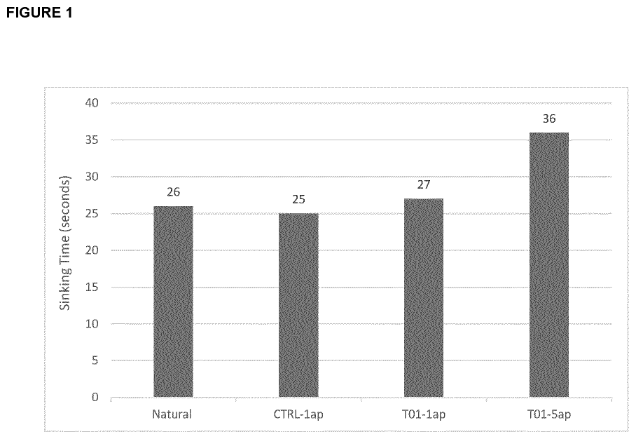

Results are presented on FIG. 1. It can be observed that after 1 application of Control (SLES 10%) the sinking time decreases when compared to the Natural group. This result reflects the damage to the hair occurred by SLES.

From the first application of the Shampoo, the hair fiber is less hydrophobic (higher sinking time) when compared to the Natural group and also to the 1 application CTRL group (10% SLES).

And the tresses of group T01_5ap, showed significantly (Dunnett test, p<0.05) higher values of sinking time when compared to the Natural group.

So, the composition according to the invention comprising a combination of LAE and a mild surfactant system, efficiently protects the hair fiber.

### Example 5: Hair Fiber Protection Study

The technique of high-resolution Scanning Electron Microscopy with emission of electrons through an application gun of an electric field allows to evaluate the protection of damage to the hair through the application of hair treatment products.

This study aimed at evaluating the protection against damage through Scanning Electron Microscopy for the shampoo according to example 2 in comparison with a naturally cleaned swatch, separated as below:
Group CTR: Swatch naturally cleaned with sodium lauryl ether sulphate (SLES 20%);
Group Shampoo: Swatch washed with the shampoo and damaged through multiple brushings;
Group Shampoo was compared with the cleaned swatch without damaging by brushing of the Group CTR.

One natural hair swatch of 20 cm and 3 grams was separated into two different groups: CTR and Shampoo.

The groups were previously treated with the control shampoo (SLES 20%) before the start of the study, so as to remove any residues from the bleaching process.

After the step of removal of the residues, the shampoo was applied on the swatches as follows:
1. The hair was rinsed for 30 seconds in running water and the excess of water was removed;
2. With the wet swatches, 0.4 mL of shampoo per gram of hair was applied on the swatches and spread by massages from the root to the ends (three times each side of the swatch);
3. After the previous step, the swatches were inserted in the brushing device and, then, brushed for 2000 times (one thousand times each side of the swatch);
4. After the first step of brushing, the cycle mentioned in items 2 and 3 were repeated two other times, until completing 6000 brushings;
5. In the end, 6 strands of each group (CTR, Shampoo) were randomly collected and sent for images acquisition with the Scanning Electron Microscope (SEM).

SEM Analysis

The images were performed with the Scanning Electron Microscope with electrons gun by field emission. The acquisition of these images has as objective to analyze the surface profile of the hair fibers after the treatments previously described: how the products protect the hair fibers from damages. For that, six strands of each swatch treated with Shampoo were randomly taken and put in aluminum “stubs” with graphite tape, so that the electrons can flow through the fibers without damaging them. The strands are then scanned by the microscope and six images (one for each strand) with amplitude of 2000× of magnification are done, highlighting the profile presented by the scanning. In addition, an illustrative image of 10000× of magnification is performed.

The damages are manually selected within the image by a trained technician and a macro, internally developed, reads and quantifies the regions of damage selected.

In the end, the damage area selected is divided by the total area analyzed and the percentage of damage for the image analyzed is quantified. The data for six swatches were analyzed statistically (Student t test, p<0.05)

Results:

The visual observation of enlarged images of each one of the treatments allows very easily to notice a fine build-up on the cuticles of the Shampoo. Its presence may be responsible for the lower value of damage observed for treatment in comparison with the naturally cleaned swatch.

The following quantitative results obtained confirm this first observation:

TABLE 5
Mean of percentage of hair damage per treatment

| Treatment | CTR | Shampoo |
|---|---|---|
| % of hair damage | 23.2% ± 2.4% | 1.6% ± 0.2% |

The Shampoo according to the invention allows significant lower damages (p<0.001) compared to CTR.

Therefore, the composition according to the present invention and comprising a combination of LAE and a specific mild surfactant system, protects the hair from the occurrence of damages.

### Example 6

CLINICAL STUDY—EVALUATION of anti-dandruff efficacy of a shampoo according to the present invention after 56 days under normal conditions of use through dermatological evaluation.

The shampoo used in the following evaluations is a shampoo according to example 2.

No subject interrupted the study, which reveals a good tolerance of the product. A very good skin and eye tolerance could be demonstrated during the 8 weeks.

Participants:

33 subjects (19 Female and 14 Male) aged between 19 and 55 years old (mean age 34±2) with moderate to severe dandruff

Protocol:
Frequency and mode of application: Apply to wet hair, massaging the scalp for 1 or 2 minutes.

Rinse and repeat the operation. In the first 15 days apply once a day. After 15 days, apply 3 times in the week.
Area: Scalp and hair.
Duration of application of the investigational product: 56 days
The first application of the product was carried out by a hairdresser expert on the subjects' scalp and hair, at the research institute, under the responsibility of the investigator.
All other applications during the study were carried out at home, under normal conditions of use.

The planning of the visits was as follows:
Visit 1: Inclusion (D1)=D1T0 value. When immediate evaluation after using the product: D1Timm value.
Period of application at home: D1 to 56
Visit 2: D15
Visit 3: D29
Visit 4: End of the study (D57)

Part 1. Antidandruff Efficacy Evaluation

1.1 Evaluation of the Scalp Oiliness Through Sebumeter® Equipment Measurements

The evaluation of the level of sebum in the scalp was performed with the Sebumeter® equipment after acclimatization for 20 minutes in a room with controlled temperature and humidity (21° C.±1° C. and 45%±5% respectively). After this period, the subject had his/her hair parted in the middle with the aid of a comb and the casual level of sebum was carried out according to the internal work instructions. The measurements were performed in triplicate (three linear measurements) in the top and central region of the scalp.

The average of the values was considered for the evaluation of oiliness.

Sebumeter® measurements were performed again in D1Timm (immediately after the first wash), D15, D29 and in D57 after acclimatization for 20 minutes in a room with controlled temperature and humidity (21° C.±1° C. and 45%±5% respectively).

Results:

The mean value of the evaluation of scalp oiliness through Sebumeter® readings was found to be significantly (Wilcoxon test, p<0.001) lower (−81.7%) after the first use of the product (D1Timm). This immediate effect is particularly beneficial for users.

The effect persists over time since at the end of the study (D57) we still observe a statistically significant decrease in the Mean Value (−32.9%) when compared to D1T0.

1.2 Clinical evaluation of the scalp through visual evaluation (evaluation of erythema) The dermatological clinical evaluation was carried out through visual assessment of the erythema of the scalp of the subjects using a five-point scale, as follows:
0=absent;
1=very mild (almost imperceptible) erythema;
2=well-defined erythema;
3=moderate to severe erythema;
4=severe erythema

For each subject, a dermatological evaluation was carried out in D1T0 (before applying the product), D1Timm, D15, D29 and in the final visit D57.

The Dermatologist should be the same throughout the study for each participant.

Results:

| Dx vs D1TO | D1Timm | D15 | D29 | D57 |
|---|---|---|---|---|
| % variation of | −34.7% | −43.1% | −34.7% | −59.9% |
| Mean Score | | | | |

The mean score of clinical evaluation of the scalp through visual evaluation (erythema evaluation) was found to be significantly (Wilcoxon test, p<0.001) lower at all timepoints, when compared to D1T0.

The composition according to the invention allows a beneficial action on the scalp with a reduction of erythema.

1.3 Clinical Evaluation of Dandruff by Quadrants Through Visual Evaluation

The clinical evaluation of dandruff on the scalp of the subjects was performed using the Squire

Method (Squire, R., & Goode, K. (2002). A randomized, single-blind, single-centre clinical trial to evaluate comparative clinical efficacy of shampoos containing ciclopirox olamine (1.5%) and salicylic acid (3%), or ketoconazole (2%, Nizoral®) for the treatment of dandruff/seborrhoeic dermatitis. Journal of Dermatological Treatment, 13(2), 51-60. doi:10.1080/095466302317584395). The subjects' scalp was divided into four imaginary quadrants and it was taken into account the affected area and the severity of dandruff in each quadrant, as follows:
The affected area within each quadrant was scored on a 5-point scale, in which:
0: <10% of the occupied area;
1: 10%-30% of the occupied area;
2: 30-50% of the occupied area;
3: 50-70% of the occupied area;
4: >70% of the occupied area.
The dandruff severity within each quadrant was scored on a 5-point scale, in which:
1: Small scales similar to a greyish white powder;
2: Intermediate;
3: Large flakes loosely adhered to the scalp forming an irregular whitish surface or small flakes partially adhered to the scalp;
4: Intermediate;
5: Flakes adhered to the scalp as white or yellowish plaques.

Finally, an overall score was calculated by multiplying the score for the affected area of each quadrant by its corresponding severity score. At the end, the scores for the four quadrants were added. It was provided an overall dandruff score between 0 and 80.

Results:

The Mean Score of the clinical evaluation of dandruff by quadrants through visual evaluation was found to be significantly (Paired t-test, p<0.001) lower at all timepoints, when compared to D1T0.

A decrease of more than 80% in Mean Score was immediately obtained (D1Timm) and then maintained at more than 90% throughout the 8 weeks even when the application of the product was spaced 3 times a week.

This efficacy is particularly interesting in the reduction or even the disappearance of the flakes.

Part 2—Evaluation of the Fiber Protection

2.1 Sensory Evaluation by Hairdresser Expert

The sensory evaluation of the subject's hair quality was carried out by a hairdresser expert using standard scales. For each subject, an evaluation was carried out in D1T0 (before applying the product) and D1Timm (after rinsing the product and drying the strands).

The shampoo was applied by the hairdresser expert and the rinse was performed in an appropriate washbasin. It was important that the hair was completely dry for further evaluation and in this way, the hairdresser was instructed to dry the subjects' hair with a hairdryer at low power (not to cause erythema), without styling with a brush and/or using a flat iron.

The evaluation of each of the parameters was through a 5-point scale. The evaluation was made in a sensorial and visual way by the expert hairdresser.

The following items has been evaluated after D1Timm (Wilcoxon test, p<0.01):
Hydration/dryness
   Significant increase of 29.1% in hydration, indicating no dryness after product use
Softness to touch
   Significant increase of 14.5% in hair softness immediately after use
Combability
   no significant change in hair combability was observed, indicating no tangling after product use
Shine
   Significant increase of 13.1% in hair shine immediately after use
Split ends
   No significant increase in split ends, indicating no damage after use

Part 3—Subjective Evaluation Questionnaire

3.1 Dermatology Life Quality Index Questionnaire—DLQI Score

All subjects answered a questionnaire to assess the impact that dandruff has on their quality of life.

Results:

| Dx vs D1TO | D15 | D29 | D57 |
|---|---|---|---|
| % variation of | −80.9% | −81.3% | −80.4% |
| Mean Score | | | |

The mean score of the ‘Dermatology Life Quality Index Questionnaire—DLQI score’ was found to be significantly (paired t-test, p<0.001) lower at all timepoints, when compared to D1T0.

These results reflect the very high level of user satisfaction with this product according to the invention in improving their quality of life by taking care of their dandruff condition.

3.2 Other Items

All subjects answered a subjective questionnaire to assess the efficacy of the product used throughout the study. The subjective questionnaire was answered in D1Timm, D15, D29 and D57.

The table below give the Mean value and the percentage of subjects who rated the composition 6 or 8.

| | D1 Timm | D57 |
|---|---|---|
| Deeply cleanses the scalp | Mean Value: 8.30 | Mean Value: 9.15 |
| | 88% rated ≥6 | 97% rated ≥6 |
| | 76% rated ≥8 | 94% rated ≥8 |
| Promotes a deep cleansing | Mean Value: 8.85 | Mean Value: 8.91 |
| sensation on the strands | 94% rated ≥6 | 94% rated ≥6 |
| | 76% rated ≥8 | 88% rated ≥8 |
| Reduces scalp itchiness | Mean Value: 7.48 | Mean Value: 8.33 |
| | 88% rated ≥6 | 94% rated ≥6 |
| | 58% rated ≥8 | 76% rated ≥8 |
| Promotes a feeling of | Mean Value: 9.39 | Mean Value: 9.55 |
| freshness | 97% rated ≥6 | 97% rated ≥6 |
| | 91% rated ≥8 | 94% rated ≥8 |
| Overall opinion about the | Mean Value: 8.67 | Mean Value: 8.47% |
| product | 91% rated ≥6 | 94% rated ≥6 |
| | 85% rated ≥8 | 72% rated ≥8 |

1. A topical composition comprising:
(a) from 0.05 to 1%, preferably from 0.09 to 0.8%, by weight of N-ethyl-lauroyl-arginate relative to the total weight of the composition;
(b) a surfactant system comprising:
(b1) one or more amino acid-based anionic surfactants;
(b2) one or more alkyl glucoside non-ionic surfactants;
(b3) one or more amphoteric surfactants different from betaine amphoteric surfactants;
(c) one or more physiologically acceptable carriers and/or adjuvants;
wherein the surfactant system does not comprise more than six surfactants.

2. The topical composition according to claim 1 wherein the surfactant system comprises:
(b1) a total amount of amino acid-based anionic surfactants ranging from 0.09 to 12%, preferably from 2 to 10%, more preferably from 5 to 8% by weight relative to the total weight of the composition;
(b2) a total amount of alkyl glucoside non-ionic surfactants ranging from 1 to 30%, preferably from 2 to 15%, more preferably from 3 to 8% by weight relative to the total weight of the composition;
(b3) a total amount of amphoteric surfactants ranging from 1.5 to 10%, preferably from 2 to 8%, more preferably from 2 to 5% by weight relative to the total weight of the composition.

3. The topical composition according to claim 1 wherein the weight ratio total amount of anionic surfactant/total amount of non-ionic surfactant is equal or superior to 1.

4. The topical composition according to claim 1 wherein the weight ratio total amount of anionic surfactant/total amount of non-ionic surfactant ranges from 1 to 2.

5. The topical composition according to claim 1 wherein the weight ratio total amount of anionic surfactant/total amount of amphoteric surfactant is equal or superior to 1.7.

6. The topical composition according to claim 1 wherein the weight ratio total amount of anionic surfactant/total amount of non-ionic surfactant is equal or superior to 1 and the weight ratio total amount of non-ionic surfactant/total amount of amphoteric surfactant is equal or superior to 1.

7. The topical composition according to claim 1 wherein the adjuvants comprise one or more, preferably only one, thickener.

8. The topical composition according to claim 1 wherein the carrier is water.

9. The topical composition according to claim 1 wherein the amino acid-based anionic surfactant is selected from the group consisting of sodium lauroyl glutamate, sodium lauroyl glycinate, sodium cocoyl glutamate, TEA-cocyl glutamate, disodium cocoyl glutamate, sodium cocoyl glycinate, potassium cocoyl glycinate, sodium cocoyl alaninate, sodium myristoyl glutamate, disodium myristoyl glutamate, sodium sarcosinate, potassium cocoyl glycinate, sodium cocoyl alaninate, sodium cocoyl apple amino acids and mixtures thereof, preferably the amino acid-based anionic surfactant is disodium cocoyl glutamate.

10. The topical composition according to claim 1 wherein the alkyl glucoside non-ionic surfactant is selected from the group consisting of decyl glucoside, arachidyl glucoside, mixture of C₁₂-C₂₀ alkyl glucoside, mixture of C₈-C₁₆ alkyl glucoside, cetearyl glucoside, ethyl glucoside, lauryl glucoside, coco glucoside, caprylyl/capryl glucoside and mixtures thereof, preferably the alkyl glucoside non-ionic surfactant is selected from the group consisting of decyl glucoside, lauryl glucoside, coco glucoside and mixtures thereof, more preferably the alkyl glucoside non-ionic surfactant is decyl glucoside.

11. The topical composition according to claim 1 wherein the amphoteric surfactant is selected from the group consisting of sultaines, alkylpolyaminocarboxylates, alkylamphoacetates, alkylamphoacetate derivatives, alkylamphodiacetates, alkylamphodiacetates derivatives, glycine derivatives and mixtures thereof, preferably the amphoteric surfactant is sodium cocoamphoacetate.

12. The topical composition according to claim 1 wherein the composition further comprises an anti-dandruff agent, the antidandruff agent being selected from the group consisting of sodium shale oil sulfonate, tea tree oil, piroctone olamine, ciclopiroxolamine, zinc pyrithione and mixtures thereof, preferably a combination of Sodium Shale Oil Sulfonate and tea tree oil.

13. The topical composition according to claim 1 wherein the composition comprises a hair fiber benefit agent, the hair fiber benefit agent being sodium dilauramidoglutamide lysine.

14. The topical composition according to claim 1 wherein the composition is in the form of a solution, lotion, foam or gel.

15. The topical composition according to claim 1 wherein the topical composition is a topical cleansing composition, a shampoo or a conditioner.
